(19) United States
(12) Patent Application Publication
(10) Pub. No.: US 2024/0300958 A1
(43) Pub. Date: Sep. 12, 2024

(54) COMPOUNDS AND THE USES AND PHARMACEUTICAL COMPOSITIONS THEREOF

(71) Applicants: BEIJING YOUZHUJU NETWORK TECHNOLOGY CO. LTD., Beijing (CN); Lemon Inc., Grand Cayman (KY)

(72) Inventors: Yuwei Yang, Los Angeles, CA (US); Hao Zhou, Beijing (CN)

(21) Appl. No.: 18/599,116

(22) Filed: Mar. 7, 2024

(30) Foreign Application Priority Data
Mar. 8, 2023 (CN) 202310218620.1

(51) Int. Cl. C07D 487/04; A61K 31/5025; A61P 11/00; A61P 35/00

(52) U.S. Cl. CPC C07D 487/04; A61K 31/5025; A61P 11/00; A61P 35/00

(57) ABSTRACT

The present invention provides a compound which is the compound represented by Formula A or a pharmaceutically acceptable salt thereof. The present invention further provides a pharmaceutical composition comprising the compound as described above and at least one pharmaceutically acceptable excipient. The present invention further provides the use of the compound as described above in the manufacture of a medicament for the treatment of at least one of inflammation, fibrotic disease and cancer. With the above aspects, the present invention provides a DDR1 inhibitor having both high inhibitory activity and high selectivity, and thus provides a novel solution for the treatment of DDR1-related diseases.

### CROSS-REFERENCE

This application claims the benefit of Chinese Patent Application No. 202310218620.1, filed on Mar. 8, 2023, which is incorporated by reference herein in its entirety.

### FIELD OF THE INVENTION

The present invention relates to the pharmaceutical field, in particular to compounds and the uses and pharmaceutical compositions thereof.

### BACKGROUND OF THE INVENTION

Receptor Tyrosine Kinases (RTKs) are the largest class of enzyme-linked receptors, and about 60 different receptor tyrosine kinases can be identified in the human genome. They are both receptors and enzymes, and are capable of binding to a ligand and phosphorylating a tyrosine residue of a target protein. RTKs generally comprise three parts: an extracellular domain containing a ligand binding site, a single transmembrane hydrophobic alpha-helical region, and an intracellular domain comprising tyrosine protein kinase activity. RTKs are cell surface receptors with high-affinity for a variety of cytokines and hormones. Mutations in RTKs will activate a series of signaling cascades that have numerous effects on protein expression. RTKs are key regulators of normal cellular processes and also play a key role in the development and progression of many types of cancer.

The DDRs family includes two subtypes, DDR1 and DDR2, which differ from other members of the RTK family in their discotic domain homologous repeats in the extracellular domain, which are also found in a variety of other transmembrane proteins and secretory proteins. There are five splicing isoforms of DDR1 (DDR1a, DDR1b, DDR1c, DDR-d and DDR1e) which differ in degree of glycosylation, protein-protein interaction, expression pattern, phosphorylation and activity, whereas DDR2 has only one structure. Common RTKs generally have a peptide-like growth factor as ligand, but DDRs are activated by various types of triple helical collagen. It is shown in studies that DDR1 and DDR2 have specific ligands, wherein DDR1 can bind to various types of collagen and DDR2 only binds to fibrous (types I, II, III) and non-fibrous (type X) collagens. Collagens in its native triple-helical form can activate DDRs while heat denatured collagens lacking the native triple-helix cannot induce kinase activity. In terms of tissue distribution, DDR1 is predominantly expressed in epithelial cells of the brain, kidney, colon, and lung, and DDR2 is expressed in mesenchymal cells including fibroblasts, myofibroblasts, smooth muscle cells, and in the heart, skin, lung, kidney, and chondrocytes in connective tissues. The activation manner of DDR1 is more distinctive. Generally, RTKs exhibit rapid activation and inactivation, while DDR1 exhibits slower activation and inactivation with prolonged phosphorylation time. Under the activation with collagen ligands, DDR1 takes about 30 minutes to be phosphorylated for a duration of up to 16 hours. Unlike the ligand-induced dimerization of most RTKs, DDRs already dimerize prior to binding to collagen, and upon binding to collagen, the kinase domain autophosphorylates as signaling proceeds to the kinase domain, leading to activation of relevant downstream signaling pathways. DDR1 has been shown to regulate a variety of cellular signaling pathways and plays a key role in regulating important processes such as cell differentiation, proliferation, adhesion, migration, invasion and matrix remodeling, however, the mechanism of extracellular collagen binding and cytoplasmic kinase domain activation of the receptor has not yet been elucidated. Similar to other RTKs, DDR1 dysregulation can cause a variety of diseases such as inflammation, neurodegenerative diseases, fibrotic diseases, atherosclerosis and cancer, etc.

In particular, the kinase domain of DDRs has a high sequence and morphological homology to Abl or c-Kit. Reported co-crystal structures of the DDR1 kinase domain with a small molecule inhibitor suggest that the DDR1 kinase domain contains loops or motifs similar to those in other RTKs, mainly including a hinge region, a catalytic loop, and an activation loop. Different splicing patterns of DDR1 gene exons resulted in 5 isoforms of DDR1 protein: DDR1a, DDR1b, DDR1c, DDR1d and DDR1e, wherein DDR1a, DDR1b and DDR1c are full-length functional receptors having kinase activities and comprising kinase domains, whereas DDR1d and DDR1e are non-functional receptors without a complete domain or lacking kinase activity. DDR1a contains 876 amino acids, DDR1b contains 913 amino acids, and DDR1c of 919 amino acids is the longest one of these isomers. Compared with DDR1a, DDR1b and DDR1c comprise the tyrosine residue Tyr513, which is important for DDR1 signaling, in the extra 37 amino acid sequences in the intracellular juxtamembrane region. DDR1d contains only 508 amino acids, and the cause of mutation may be the deletion of exons 11 and 12, resulting in the generation of a stop codon and the premature termination of transcription. DDR1e contains 767 residues, even having the KD part, it lacks an ATP-binding site and therefore will not autophosphorylate, making DDR1e an inactive receptor. DDR2 has only one isoform consisting of 855 amino acids and having kinase activity. DDRs all need to bind to different types of collagen to initiate downstream pathways. It should be noted that only active collagens, i.e., triple-helix collagens, can bind to DDRs, while thermally denatured collagens (such as gelatin, etc.) cannot be recognized or bound by DDRs. There are 28 different types of collagen in vertebrates, named with Roman numerals I to XXVIII, and can be divided into two main categories: fibrous type and non-fibrous type. Fibrous collagens are mainly consisted of type I, type II, type III, type V, type XI, type XXIV and type XXVII, while the others belong to non-fibrous collagens. DDRs all bind to fibrillar collagens with certain ligand specificity. DDR1 preferentially binds to collagens I-V and VIII, while DDR2 binds to collagens I-III, V and X. In addition, DDR1 can also bind to periostin, another component in the ECM.

In human breast and colon cancer cell lines, DDR1 can activate MAPK and PI3K/Akt signaling pathway under p53 induction, leading to the up-regulation of the expression of anti-apoptotic protein Bcl-xl and improving cell survival under genotoxic stress conditions. In human breast cancer cells, DDR1 activation can also increase NF-KB DNA binding activity and cyclooxygenase-2 (COX-2) expression, leading to enhanced chemoresistance. DDR1 can also form a complex with Notch1, and in colon cancer cells, DDR1 activation induces the cleave of Notch1 by γ-secretase, and the resulting intracellular domain of the Notch1 is transferred to the nucleus, upregulating the expression of pro-survival genes Hes1 and Hey2. In NIH3T3 fibroblasts and MCF7 breast cancer cells, DDR1 inhibits cell spreading via interaction with NMHC-IIA, a contractile protein involved in cell spreading. In oral squamous carcinoma cells, as E-cadherin enters the intercellular space, DDR1 binds to the PaR3/Par6 protein to form a complex that is recruited by RhoE. Elimination of RhoA/ROCK activity and reduction of actomyosin levels at cell junctions increases the invasiveness of oral squamous carcinoma cells. In non-malignant breast cancer epithelial cells, Wnt-5a may inhibit migration through activating DDR1, and in breast cancer cell lines MCF7 and MDA-MB-231, DDR1 significantly inhibits migration only when co-expressed with phosphorylated protein DARPP32. Collagen can also bind to integrins to activate certain signaling pathways. Synergy with DDR1 activation appears in various downstream signaling cascades. In pancreatic cancer cell lines, DDR1 is activated simultaneously with integrin B1, which initiates the downstream FAK/p130CAS/JNK pathway and upregulates N-cadherin expression, leading to cell dispersion or epithelial-mesenchymal transition (EMT). DDR2 activates important signaling components including ShcA, JAK, ERK1/2 and PI3K, in a pathway similar to DDR1. DDR2 sometimes exhibits crosstalk between other cell surface receptors (other RTKs and integrin receptors), leading to diversification of downstream signaling. In a phosphoproteomic screen for type I collagen-induced DDR2 signaling, 45 downstream signaling effectors have been identified, including novel protein substrates such as Lyn, SHIP-2 and PLCL2. These signaling pathways are independent of the collagen-activated integrin signaling pathway and are specific for the DDR2 pathway.

Dysregulation of DDR1 receptor expression is closely associated with a variety of human diseases, including inflammation (hepatitis, periostitis, nephritis), cancer (glioma, non-small cell lung cancer, hepatocellular carcinoma and breast cancer, etc.), and fibrotic diseases (liver fibrosis, kidney fibrosis and lung fibrosis).

DDR1 inhibitors can be divided into two classes based on action sites: the first class of inhibitors are mainly monoclonal antibodies acting on the extracellular domain, which block the phosphorylation process of DDR1 by preventing collagen from binding to DDR1; the second class of inhibitors act on the intracellular kinase domain, affecting downstream signaling pathways. DDR1 inhibitors are often classified according to their selectivity and can be divided into multi-target inhibitors and selective inhibitors.

Despite a large number of reported DDR1 inhibitors, there are still no highly selective DDR1 inhibitors under clinical research. Multi-target kinase inhibitors may cause hypertension, proteinuria, rash, etc. It is of great significance to develop DDR1 inhibitors with both high inhibitory activity and high selectivity (or selective inhibition of several targets).

### SUMMARY OF THE INVENTION

The present invention aims to provide a DDR1 inhibitor having both high inhibitory activity and high selectivity.

In order to achieve the above goal, the present invention provides a compound which is the compound represented by Formula A or a pharmaceutically acceptable salt thereof:

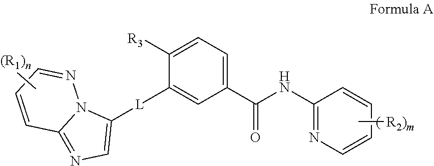

wherein each R₁ is independently alkyl, alkenyl, alkynyl, cycloalkyl, cycloalkenyl, cycloalkynyl, aryl, heterocyclyl or heteroaryl; n is 1 or 2; m is 0, 1 or 2; each R₂ is independently alkyl, haloalkyl, alkenyl, alkynyl, cycloalkyl, cycloalkenyl, cycloalkynyl, aryl, heterocyclyl and heteroaryl; R₃ is methyl, ethyl, n-propyl or isopropyl; L is ethynylene, phenylene or methylenephenyl.

The present invention further provides a pharmaceutical composition comprising a compound as described above and at least one pharmaceutically acceptable excipient.

The present invention further provides the use of a compound as described above in the manufacture of a medicament for the treatment of a disease, wherein the disease is at least one of inflammation, fibrotic disease and cancer.

With the above technical solutions, the present invention provides a DDR1 inhibitor having both high inhibitory activity and high selectivity, and thus provides a novel solution for the treatment of DDR1-related diseases.

Additional features and advantages of the present invention will be set forth in the detailed description which follows.

### BRIEF DESCRIPTION OF THE DRAWINGS

The drawings are provided for a further understanding of the present invention and constitute a part of the specification. They are used to explain the present invention together with the following specific embodiments but are not regarded as limitations to the present invention. Of the drawings:

FIG. 1 is a molecular structural model of compound C1.

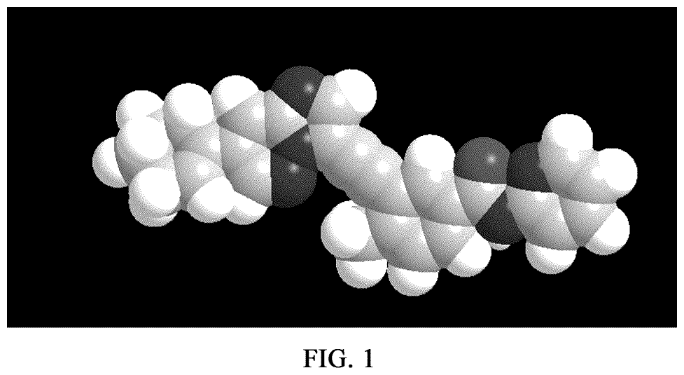

FIG. 2 is a scheme for the synthesis of compound C1.

FIG. 3 is a kinase inhibition profile of compound C1.

FIG. 4 is the inhibitory effect of compound C1 on phosphorylated DDR1 (p-DDR1) in a cellular model.

FIG. 5 is the inhibitory effect of compound C1 on DDR1-induced lung fibrosis in a cellular model.

### DETAILED DESCRIPTION

Hereinafter, specific embodiments of the present invention will be described in detail. It should be understood that the specific embodiments described herein are for illustrative and explanatory purpose only and are not limitations to the present invention.

The present invention provides a compound which is the compound represented by Formula A or a pharmaceutically acceptable salt thereof.

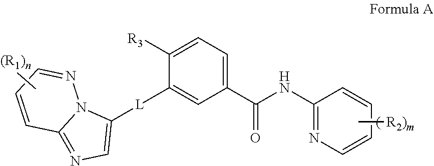

wherein each R₁ is independently alkyl, alkenyl, alkynyl, cycloalkyl, cycloalkenyl, cycloalkynyl, aryl, heterocyclyl or heteroaryl; n is 1 or 2; m is 0, 1 or 2; each R₂ is independently alkyl, haloalkyl, alkenyl, alkynyl, cycloalkyl, cycloalkenyl, cycloalkynyl, aryl, heterocyclyl and heteroaryl; R₃ is methyl, ethyl, n-propyl or isopropyl; L is ethynylene, phenylene or methylenephenyl.

Preferably, in the above embodiment, n is 1.

Preferably, in the above embodiment, R₁ is cycloalkenyl which is cyclopentenyl, methylcyclopentenyl or cyclohexenyl.

Preferably, in the above embodiment, the cycloalkenyl is cyclohexen-1-yl.

Preferably, in the above embodiment, m is 0.

Preferably, in the above embodiment, L is ethynylene.

Preferably, in the above embodiment, the compound is any one of the compounds as shown in the following Formulas C1-C12:

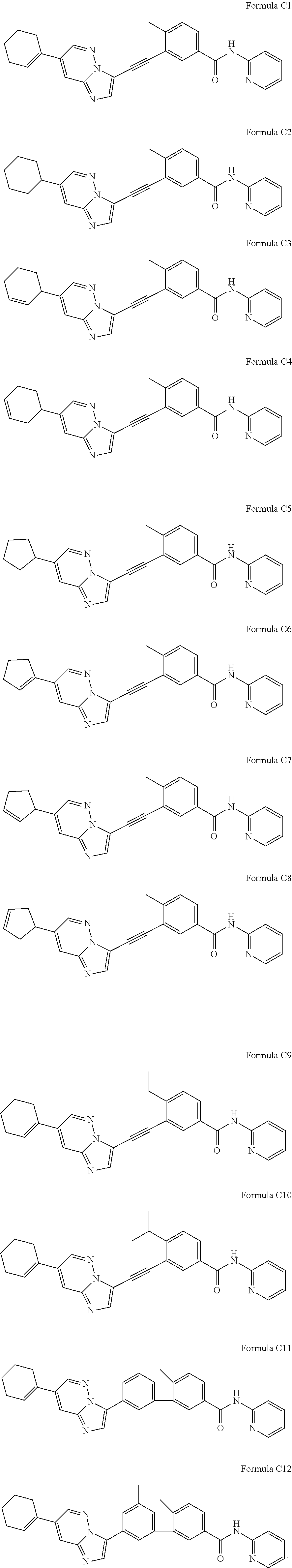

The present invention further provides a pharmaceutical composition comprising a compound as described above and at least one pharmaceutically acceptable excipient.

The present invention further provides the use of a compound as described above in the manufacture of a medicament for the treatment of a disease, wherein the disease is at least one of inflammation, fibrotic disease and cancer.

Preferably, in the above embodiment, the inflammation is at least one of hepatitis, periostitis and nephritis; the cancer is at least one of head and neck cancer, liver cancer, prostate cancer, lymphoma, leukemia, glioma, lung cancer, brain cancer, and breast cancer; and the fibrotic disease is at least one of liver fibrosis, kidney fibrosis and lung fibrosis.

The invention is illustrated in further detail by the following examples. The starting materials used in the examples are commercially available, unless otherwise specified.

### Preparation Example 1

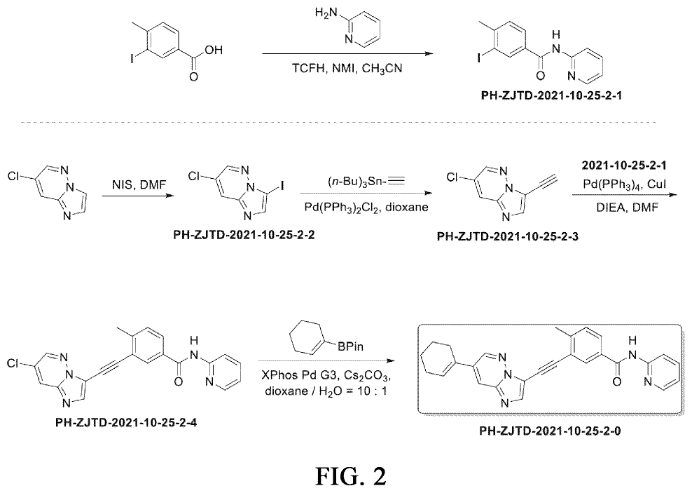

In this preparation example, the compound of formula C1 is prepared with reference to the process shown in FIG. 2.

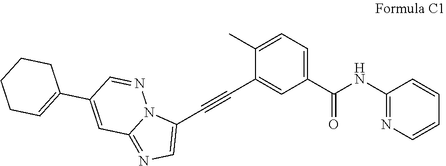 [SMILES: Cc1ccc(C(=O)Nc2ccccn2)cc1C#Cc1cnc2cc(C3=CCCCC3)cnn12]

### Synthesis of 3-iodo-4-methyl-N-(pyridin-2-yl)benzamide

With stirring, to a solution of 3-iodo-4-methylbenzoic acid (100 mg, 0.382 mmol, 1 eq.) and 2-aminopyridine (43.10 mg, 0.458 mmol, 1.2 eq.) in CH₃CN (5 mL) was added tetramethylchlorourea hexafluorophosphate (TCFH) (117.78 mg, 0.420 mmol, 1.1 eq.) and 1-methyl-1H-imidazole (97.13 mg, 1.184 mmol, 3.1 eq.) at room temperature under argon atmosphere. The reaction was continued for 6 hours at room temperature. The resulting product was centrifuged under vacuum and the precipitate was purified on a silica gel column to give 3-iodo-4-methyl-N-(pyridin-2-yl)benzamide as a white solid (100 mg, 77.49% yield). Results of mass spectrometry and nuclear magnetic resonance detection are: MS: m/z=339.00 [M+H]+; 1H NMR (400 MHZ, Chloroform-d) δ 8.83 (s, 1H), 8.42-8.39 (m, 2H), 8.30-8.29 (m, 1H), 7.85-7.78 (m, 2H), 7.37-7.35 (m, 1H), 7.12-7.09 (m, 1H), 2.50 (s, 3H).

### Synthesis of 7-chloro-3-iodoimidazo[1,2-b]pyridazine

With stirring, to a solution of 7-chloroimidazo[1,2-b]pyridazine (1 g, 6.512 mmol, 1 eq.) in DMF (10 mL) was added 1-iodopyrrolidine-2,5-dione (2.20 g, 9.768 mmol, 1.5 eq.) at room temperature under argon atmosphere. The reaction was carried out for 16 h. The resulting product was quenched with water (100 mL) and extracted with ethyl acetate (3×200 mL). The resulting organic phases were combined and washed with brine (3×200 mL), dried over anhydrous sodium sulfate and filtered. The filtrate was concentrated under a reduced pressure and the concentrated product was purified by a silica gel column to give 7-chloro-3-iodoimidazo[1,2-b]pyridazine as a white solid (1.6 g, 88% yield). Results of mass spectrometry and nuclear magnetic resonance detection are: MS: m/z=279.90 [M+H]+; 1H NMR (400 MHZ, DMSO-d6) δ 8.77 (d, J=2.4 Hz, 1H), 8.45 (d, J=2.4 Hz, 1H), 7.94 (s, 1H).

### Synthesis of 7-chloro-3-ethynylimidazo[1,2-b]pyridazine

With stirring, to a solution of 7-chloro-3-iodoimidazo[1,2-b]pyridazine (1.6 g, 5.726 mmol, 1 eq.) and tributyl(ethynyl)stannane (2.71 g, 8.589 mmol, 1.5 eq.) in dioxane (10 mL) was added Pd(PPh₃)₂Cl₂ (200.92 mg, 0.286 mmol, 0.05 eq.) at room temperature under argon atmosphere. The reaction was carried out at 90° C. for 2 hours. The reaction product was concentrated under a reduced pressure and the concentrated product was purified by a silica gel column to give 7-chloro-3-iodoimidazo[1,2-b]pyridazine as a yellow solid (500 mg, 49% yield). Results of mass spectrometry and nuclear magnetic resonance detection are: MS: m/z=178.05 [M+H]+. 1H NMR (300 MHZ, Chloroform-d) § 8.43 (d, J=2.4 Hz, 1H), 8.06-7.98 (m, 2H), 3.81 (s, 1H).

### Synthesis of 3-(2-{7-chloroimidazo[1,2-b]pyridazin-3-yl}ethynyl)-4-methyl-N-(pyridin-2-yl)benzamide

With stirring, to a solution of 3-iodo-4-methyl-N-(pyridin-2-yl)benzamide (120 mg, 0.357 mmol, 1.0 eq) and 7-chloro-3-ethynylimidazo[1,2-b]pyridazine (75.63 mg, 0.426 mmol, 1.2 eq.) in DMF (6 mL) was added Pd(PPh₃)₄ (20.49 mg, 0.018 mmol, 0.05 eq.), CuI (5.07 mg, 0.027 mmol, 0.075 eq.) and DIEA (91.74 mg, 0.708 mmol, 2 eq.). The reaction was carried out at 90° C. for 2 hours. The reaction product was concentrated under a reduced pressure and the concentrated product was purified by a silica gel column to give a yellow solid, namely 3-(2-{7-chloroimidazo[1,2-b]pyridazin-3-yl}ethynyl)-4-methyl-N-(pyridin-2-yl)benzamide (90 mg, 66.7% yield).

### Synthesis of compound of Formula C1, namely 3-{2-[7-(cyclohex-1-en-1-yl)imidazo[1,2-b]pyridazin-3-yl]ethynyl}-4-methyl-N-(pyridin-2-yl)benzamide

With stirring, to a mixture of 3-(2-{7-Chloroimidazo[1,2-b]pyridazin-3-yl}ethynyl)-4-methyl-N-(pyridin-2-yl)benzamide (75 mg, 0.192 mmol, 1 eq.), 2-(cyclohex-1-en-1-yl)-4,4,5,5-tetramethyl-1,3,2-dioxaborane (60.36 mg, 0.288 mmol, 1.5 eq.), dioxane (4 mL) and water (0.4 mL) was added XPhos Pd G3 (8.19 mg, 0.012 mmol, 0.05 eq.) and Cs₂CO₃ (126.03 mg, 0.384 mmol, 2 eq.). The reaction was carried out with stirring at 130° C. under microwave for 1 hour. The reaction product was concentrated under a reduced pressure and the concentrated product was purified by a silica gel column to give a light yellow solid, namely 3-{2-[7-(cyclohex-1-en-1-yl)imidazo[1,2-b]pyridazin-3-yl]ethynyl}-4-methyl-N-(pyridin-2-yl)benzamide. Results of mass spectrometry and nuclear magnetic resonance detection are: MS: m/z=434.20 [M+H]+. 1H NMR (400 MHZ, DMSO-d6) δ 10.91 (s, 1H), 8.99 (s, 1H), 8.41-8.40 (m, 1H), 8.23-8.17 (m, 3H), 8.04-7.98 (m, 2H), 7.88-7.83 (m, 1H), 7.51 (d, J=8.4 Hz, 1H), 7.20-7.17 (m, 1H), 6.70 (s, 1H), 2.67 (s, 3H), 2.50-2.46 (m, 2H), 2.33-2.27 (m, 2H), 1.78-1.75 (m, 2H), 1.65-1.62 (m, 2H).

### Preparation Examples 2-12

The compounds of Formulas C2 to C12 were prepared with reference to Preparation Example 1.

### Test Example 1

This test example evaluates the inhibitory activities of C1-C12 compounds on DDR1.

The purchasing information of raw materials is as follows: DDR1 (Carna, No 08-113, Lot. No PO1181112140018); FAM-P13 peptide (GL Biochem, Cat. No. 114204, Lot. No. P100804-ZJ114204; ATP (Sigma, Cat. No. A7699-1G, CAS No. 987-65-5); DMSO (Sigma, Cat. No. D2650, Lot. No. 474382); EDTA (Sigma, Cat. No. E5134, CAS No. 60-00-4); Staurosporine (Selleckchem, Cat. No. S1421, Lot. No. S142105).

1× kinase base buffer (50 mM HEPES, pH 7.5, 0.0015% Brij-35) and a stop buffer (100 mM HEPES, pH 7.5, 0.015% Brij-35, 0.2% Coating Reagent #3, 50 mM EDTA) were prepared for testing kinases.

The compounds were diluted with 100% DMSO to 100 times the highest final inhibitor concentration required in the reaction. 100 μl of dilution of the compound was transferred to wells of a 96-well plate. For example, 100000 nM solution of the compound in DMSO was prepared in this step in case that an inhibitor concentration of 1000 nM is required. 100 μl of 100% DMSO was added to two empty wells of a 96-well plate for no compound control and no enzyme control. This plate was labeled as the source plate. A new 96-well plate with 5 μl of compound transferred from the source plate is used as an intermediate plate. 95 μl of 1× kinase buffer was added to each well of the intermediate plate. The compounds in the intermediate plate was mixed on a shaker for 10 minutes.

5 μl of sample in each well from the 96-well intermediate plate was transferred to the 384-well plate in duplicate. For example, well A1 of the 96-well plate was transferred to wells A2 and A1 of the 384-well plate, and well A2 of the 96-well plate was transferred to wells A3 and A4 of the 384-well plate, and so forth. 2.5× enzyme solution was prepared: 1× kinase basal buffer with added kinase. 2.5× peptide solution was prepared: 1× kinase basal buffer with added FAM-labeled peptide and ATP. To an assay plate already contained 5 μl of 10% dimethyl sulfoxide compound, 2.5× enzyme solution was transferred, and each well of the 384-well assay plate was added 10 μl of 2.5× enzyme solution. After incubation at room temperature for 10 minutes, 2.5× peptide solution was transferred to the assay plate. Each well of the 384-well assay plate was added 10 μl of 2.5× peptide solution. Kinase reaction and termination: incubation at 28° C. for a specified period followed by the addition of 25 μl stop buffer to stop the reaction. The data was read and collected. The data were fitted to obtain IC50 values, and the results are shown in Table 1.

TABLE 1

| | | DDR1 |
|---|---|---|
| | | IC50 |
| Compound | Structural formula | (nM) |
| C1 | 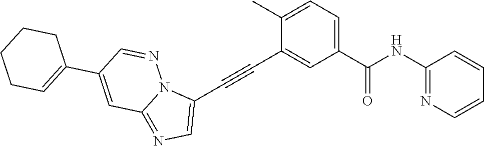 [SMILES: Cc1ccc(C(=O)Nc2ccccn2)cc1C#Cc1cnc2cc(C3=CCCCC3)cnn12] | 23 |
| C2 | 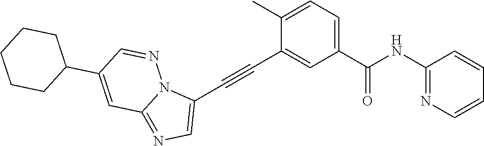 [SMILES: Cc1ccc(C(=O)Nc2ccccn2)cc1C#Cc1cnc2cc(C3CCCCC3)cnn12] | <500 |
| C3 | 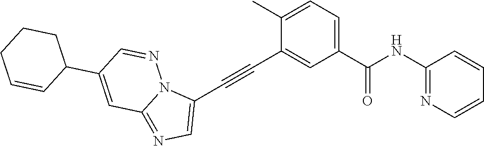 [SMILES: Cc1ccc(C(=O)Nc2ccccn2)cc1C#Cc1cnc2cc(C3C=CCCC3)cnn12] | <500 |
| C4 | 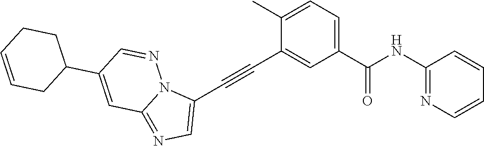 [SMILES: Cc1ccc(C(=O)Nc2ccccn2)cc1C#Cc1cnc2cc(C3CC=CCC3)cnn12] | <500 |
| C5 | 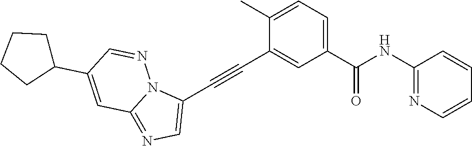 [SMILES: Cc1ccc(C(=O)Nc2ccccn2)cc1C#Cc1cnc2cc(C3CCCC3)cnn12] | <500 |
| C6 | 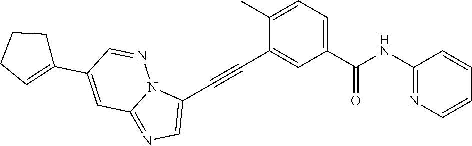 [SMILES: Cc1ccc(C(=O)Nc2ccccn2)cc1C#Cc1cnc2cc(C3=CCCC3)cnn12] | <500 |
| C7 | 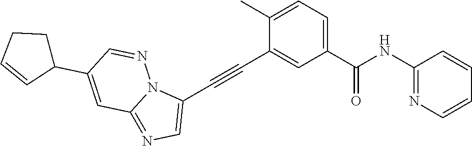 [SMILES: Cc1ccc(C(=O)Nc2ccccn2)cc1C#Cc1cnc2cc(C3C=CCC3)cnn12] | <500 |
| C8 | 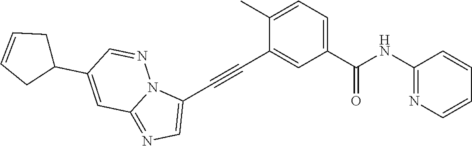 [SMILES: Cc1ccc(C(=O)Nc2ccccn2)cc1C#Cc1cnc2cc(C3CC=CC3)cnn12] | <500 |
| C9 | 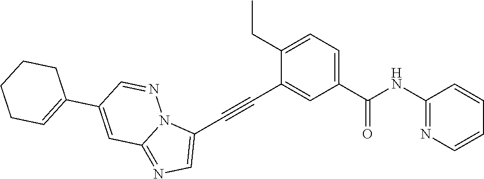 [SMILES: CCc1ccc(C(=O)Nc2ccccn2)cc1C#Cc1cnc2cc(C3=CCCCC3)cnn12] | <500 |
| C10 | 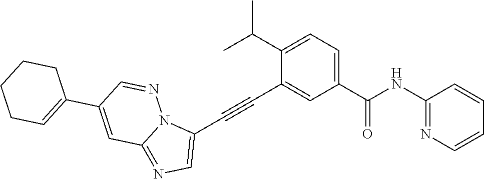 [SMILES: CC(C)c1ccc(C(=O)Nc2ccccn2)cc1C#Cc1cnc2cc(C3=CCCCC3)cnn12] | <500 |
| C11 | 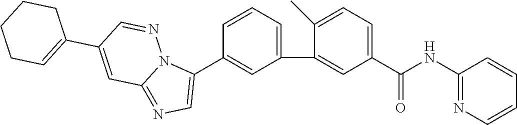 [SMILES: Cc1ccc(C(=O)Nc2ccccn2)cc1-c1cccc(-c2cnc3cc(C4=CCCCC4)cnn23)c1] | <500 |
| C12 | 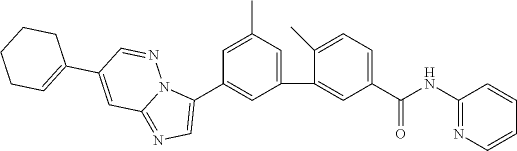 [SMILES: Cc1cc(-c2cc(C(=O)Nc3ccccn3)ccc2C)cc(-c2cnc3cc(C4=CCCCC4)cnn23)c1] | <500 |
| Staurosporine | 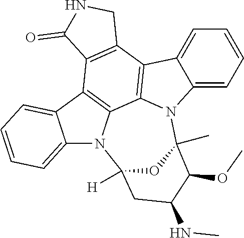 [SMILES: CN[C@H]1C[C@@H]2O[C@](C)([C@H]1OC)n1c3ccccc3c3c4c(c5c6ccccc6n2c5c31)C(=O)NC4] | 1.52 |
| DC1 | 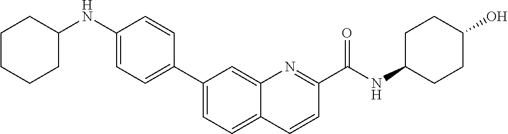 [SMILES: O=C(N[C@H]1CC[C@H](O)CC1)c1ccc2ccc(-c3ccc(NC4CCCCC4)cc3)cc2n1] | >1000 |

The data in Table 1 shows that compounds C1-C12 exhibit inhibitory activities on DDR1, specifically, compound C1 has a strong inhibitory effect on DDR1 (with a IC50 value of 23 nM).

### Test Example 2

This Test Example evaluates the kinase inhibition profile of compound C1.

HTRF kinase assay: 2×ATP and substrate solution as well as 2× kinase and metal solution were prepared with assay buffer. 25 nL of compound was transferred to 384-well assay plate by Echo 655. After centrifugation, 2.5 μL of 2× kinase/metal ion solution was added to the 384-well assay plate followed by incubation at 25° C. for 10 min. 2.5 μL of 2× substrate and ATP solution was added to the wells followed by incubation at 25° C. for 60 min. 2× XL665 and antibody solution was prepared with assay buffer. 5 μL of kinase detection reagent was added to the wells followed by incubation at 25° C. for 60 min. Fluorescence signals at 620 nm (Cryptate) and 665 nm (XL665) were read with a microtiter plate reader.

ADP Glo kinase assay: 2×ATP and substrate solution as well as 2× kinase and metal solution were prepared with assay buffer. 20 nL of compound was transferred to 384-well assay plate by Echo 655. 2 μL of 2× kinase/metal ion solution was added followed by incubation at 25° C. for 10 min in the 384-well assay plate. 2 μL of 2× substrate and ATP solution was added to the wells followed by incubation at 25° C. for 60 min. 4 μL of ADP Glo reagent was added to the wells followed by incubation at 25° C. for 40 min. 8 μL of kinase test reagent was added to the wells followed by incubation at 25° C. for 40 min. The luminescence signals were recorded on a microtiter plate reader.

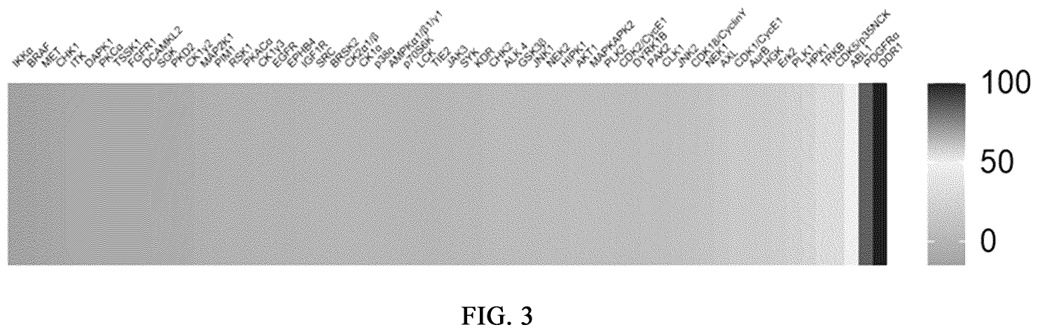

Data analysis: the reaction control (0.5% DMSO) reading was set to 0% inhibition, and the background (10 μM positive control) reading was set to 100% inhibition, then the percent inhibition for each test solution was calculated and the results are shown in Table 2 and FIG. 3.

TABLE 2

| | | 1 uM | 1 uM | | 1 uM |
|---|---|---|---|---|---|
| | | Compound C1 | Compound C1 | | Compound C1 |
| | | % Inhibition | % Inhibition | | Average |
| | | (First | (Second | | inhibition |
| No. | Kinase | measurement) | measurement) | STDEV | (%) |
| 1 | DDR1 | 99.44 | 99.12 | 0.23 | 99.28 |
| 2 | PDGFRα | 84.06 | 84.83 | 0.54 | 84.44 |
| 3 | ABL1 | 43.23 | 50.74 | 5.31 | 46.99 |
| 4 | CDK5/p35NCK | 31.19 | 31.23 | 0.03 | 31.21 |
| 5 | TRKB | 20.76 | 40.42 | 13.90 | 30.59 |
| 6 | HPK1 | 23.88 | 24.70 | 0.58 | 24.29 |
| 7 | PLK1 | 24.12 | 20.57 | 2.51 | 22.35 |
| 8 | Erk2 | 20.22 | 19.29 | 0.66 | 19.75 |
| 9 | HGK | 18.51 | 16.97 | 1.09 | 17.74 |
| 10 | AurB | 19.69 | 13.34 | 4.49 | 16.52 |
| 11 | CDK1/CycE1 | 11.90 | 20.14 | 5.83 | 16.02 |
| 12 | AXL | 15.13 | 13.31 | 1.29 | 14.22 |
| 13 | NEK1 | 14.56 | 8.97 | 3.95 | 11.76 |
| 14 | CDK18/CyclinY | 17.98 | 2.55 | 10.91 | 10.26 |
| 15 | JNK2 | 9.39 | 9.07 | 0.23 | 9.23 |
| 16 | CLK1 | 2.98 | 12.35 | 6.62 | 7.67 |
| 17 | PAK2 | 2.23 | 12.12 | 6.99 | 7.17 |
| 18 | DYRK1B | 4.14 | 9.03 | 3.46 | 6.58 |
| 19 | CDK2/CycE1 | 0.53 | 12.60 | 8.53 | 6.56 |
| 20 | PLK2 | 10.64 | 2.02 | 6.09 | 6.33 |
| 21 | MAPKAPK2 | 15.32 | −3.28 | 13.15 | 6.02 |
| 22 | AKT1 | 7.17 | 4.56 | 1.84 | 5.87 |
| 23 | HIPK1 | 6.91 | 4.17 | 1.94 | 5.54 |
| 24 | NEK2 | 8.18 | 2.89 | 3.74 | 5.54 |
| 25 | JNK1 | 4.56 | 5.69 | 0.79 | 5.13 |
| 26 | GSK3β | 0.83 | 7.83 | 4.95 | 4.33 |
| 27 | ALK 4 | −6.24 | 14.75 | 14.84 | 4.25 |
| 28 | CHK2 | 0.35 | 7.34 | 4.95 | 3.85 |
| 29 | KDR | 2.42 | 1.61 | 0.57 | 2.01 |
| 30 | SYK | 5.21 | −2.04 | 5.13 | 1.59 |
| 31 | JAK3 | −1.28 | 2.46 | 2.65 | 0.59 |
| 32 | TIE2 | −5.65 | 6.20 | 8.38 | 0.28 |
| 33 | LCK | 7.39 | −6.87 | 10.08 | 0.26 |
| 34 | p70S6K | 0.46 | −0.19 | 0.46 | 0.14 |
| 35 | AMPKαl/β1/γ1 | 2.13 | −1.96 | 2.89 | 0.09 |
| 36 | p38α | 3.39 | −3.27 | 4.71 | 0.06 |
| 37 | CK1α | 5.80 | −5.89 | 8.26 | −0.04 |
| 38 | CK2α1/β | −5.19 | 5.01 | 7.21 | −0.09 |
| 39 | BRSK2 | 0.46 | −0.94 | 0.99 | −0.24 |
| 40 | SRC | −0.76 | −1.13 | 0.26 | −0.94 |
| 41 | IGF1R | −0.05 | −2.29 | 1.59 | −1.17 |
| 42 | EPHB4 | 3.80 | −8.27 | 8.53 | −2.24 |
| 43 | EGFR | −6.86 | 1.78 | 6.11 | −2.54 |
| 44 | CK1γ3 | −7.05 | 1.91 | 6.33 | −2.57 |
| 45 | PKACα | −4.44 | −1.87 | 1.81 | −3.15 |
| 46 | RSK1 | 0.73 | −7.90 | 6.10 | −3.59 |
| 47 | PIM1 | −3.17 | −4.27 | 0.77 | −3.72 |
| 48 | MAP2K1 | −5.72 | −3.53 | 1.55 | −4.62 |
| 49 | CK1γ2 | −6.26 | −6.88 | 0.44 | −6.57 |
| 50 | PKD2 | −7.86 | −6.58 | 0.90 | −7.22 |
| 51 | SGK | −8.79 | −6.61 | 1.54 | −7.70 |
| 52 | DCAMKL2 | −8.91 | −8.39 | 0.37 | −8.65 |
| 53 | FGFR1 | 0.18 | −18.23 | 13.02 | −9.02 |
| 54 | TSSK1 | −15.13 | −3.43 | 8.28 | −9.28 |
| 55 | PKCα | −1.67 | −17.21 | 10.99 | −9.44 |
| 56 | DAPK1 | −14.48 | −5.25 | 6.53 | −9.87 |
| 57 | ITK | −3.82 | −19.16 | 10.85 | −11.49 |
| 58 | CHK1 | −10.57 | −13.61 | 2.15 | −12.09 |
| 59 | MET | −11.90 | −15.85 | 2.79 | −13.88 |
| 60 | BRAF | −6.73 | −23.94 | 12.17 | −15.33 |
| 61 | IKKα | −18.00 | −12.92 | 3.59 | −15.46 |

The data in Table 2 and FIG. 3 shows that compound C1 can inhibit DDR1 with higher selectivity.

Compounds C2-C12 can also inhibit DDR1 with higher selectivity.

### Test Example 3

This Test Example evaluated the inhibitory effect of compound C1 on phosphorylated DDR1 (p-DDR1) in a cellular model.

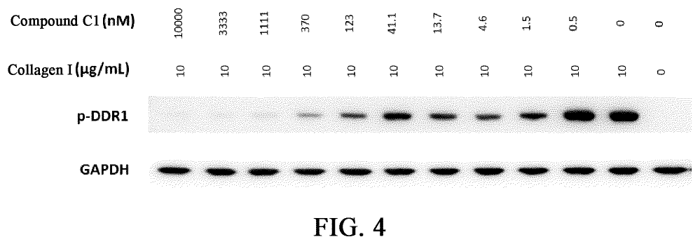

Human breast ductal carcinoma cell line T47D cells were inoculated in a 6-well plate and incubated overnight at 37° C. and 5% CO₂. After the cells are prepared, 2 μL of Compound C1 (DMSO solution of gradient concentrations) was added to a cell plate which was incubated at 37° C. and 5% CO₂ for 24 hours. Rat tail collagen (capable of stimulating DDR1 phosphorylation) was added to the cell plate followed by incubation for 2 hours at 37° C. and 5% CO₂. Harvest of cell lysates: culture medium was aspirated as much as possible at the end of the culture until the wells were almost dry. 200 μL of ice-cold lysis buffer was added. Lysate was collected into a 1.5 mL eppendorf tube through scraping. Brief vortex was followed by spin at 12000 rpm for 15 minutes in a refrigerated centrifuge. 5× protein electrophoresis loading buffer was added and then the sample was boiled. The sample supernatant was added to a SDS-PAGE gel, followed by electrophoresis, electrotransfer, blocking, primary antibody (anti-phospho-DDR1 antibody and anti-GAPDH antibody) incubation, washing, secondary antibody incubation, washing, and fluorescence development. The results are shown in Table 3 and FIG. 4.

TABLE 3

| | | | Ratio of | |
|---|---|---|---|---|
| Concentration of | GAPDH | p-DDR1 | p-DDR1 | |
| compound C1 nM | signal | signal | to GAPDH | % Inhibition |
| 10000 | 1563210 | 14730 | 0.0094 | 100.00 |
| 3333 | 1498770 | 27360 | 0.0183 | 98.66 |
| 1111 | 1428570 | 51870 | 0.0363 | 95.91 |
| 370 | 1320930 | 164340 | 0.1244 | 82.52 |
| 123 | 1486290 | 313140 | 0.2107 | 69.40 |
| 41.1 | 1407810 | 583470 | 0.4145 | 38.42 |
| 13.7 | 1383900 | 417870 | 0.3020 | 55.52 |
| 4.6 | 1578330 | 350490 | 0.2221 | 67.67 |
| 1.5 | 1495200 | 490770 | 0.3282 | 51.53 |
| 0 | 1668480 | 1113090 | 0.6671 | 0.00 |

The data in Table 3 and FIG. 4 shows that compound C1 can inhibit the phosphorylation of DDR1 at the cellular level and thus suppress tumors (e.g., breast cancers).

Compounds C2-C12 can also inhibit the phosphorylation of DDR1 at the cellular level and thus suppress tumors (e.g., breast cancers).

### Test Example 4

This Test Example evaluated the inhibitory effect of compound C1 on DDR1-mediated lung fibrosis in a cellular model.

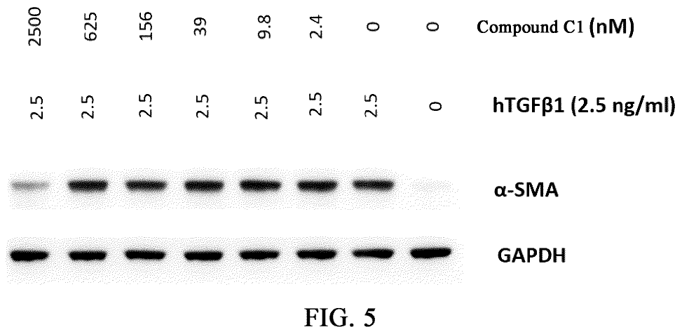

Human embryonic lung fibroblast MRC-5 cells were inoculated in a 6-well plate and incubated overnight at 37° C. and 5% CO₂. 2 μL of compound C1 (gradient concentration in DMSO solution) was added to a cell plate with prepared cells, and was incubated overnight at 37° C. and 5% CO₂. hTGFβ1 (which can stimulate DDR1-mediated lung fibrosis manifested by up-regulation of α-smooth muscle actin (α-SMA) expression) was added to the cell plate and incubated at 37° C. and 5% CO₂ for 2 hours. Harvest of cell lysates: culture medium was aspirated as much as possible at the end of the culture until the wells were almost dry. 200 μL of ice-cold lysis buffer was added. Lysate was collected into a 1.5 mL eppendorf tube through scraping. Brief vortex was followed by spinning at 12000 rpm for 15 minutes in a refrigerated centrifuge. 5× protein electrophoresis loading buffer was added and then the sample was boiled. The sample supernatant was added to a SDS-PAGE gel, followed by electrophoresis, electrotransfer, blocking, primary antibody (anti-α-SMA antibody and anti-GAPDH antibody) incubation, washing, secondary antibody incubation, washing, and fluorescence development. The results are shown in Table 4 and FIG. 5.

TABLE 4

| | Concentration | | | | |
|---|---|---|---|---|---|
| | of compound | | | | |
| hTGFβ1 | C1 | GAPDH | | Ratio of α-SMA | |
| ng/ml | nM | signal | α-SMA signal | to GAPDH | % Inhibition |
| 2.5 | 2500.00 | 35244 | 4686 | 0.1330 | 11.7380 |
| 2.5 | 625.00 | 1153680 | 629772 | 0.5459 | 48.1920 |
| 2.5 | 156.25 | 1096788 | 1353660 | 1.2342 | 108.9593 |
| 2.5 | 39.06 | 1117083 | 1304919 | 1.1681 | 103.1277 |
| 2.5 | 9.77 | 1190244 | 1386165 | 1.1646 | 102.8149 |
| 2.5 | 2.44 | 1197306 | 1390356 | 1.1612 | 102.5175 |
| 2.5 | 0.00 | 1145001 | 1296966 | 1.1327 | 100.0000 |
| 0 | 0.00 | 1443981 | 138270 | 0.0958 | — |

The data in Table 4 and FIG. 5 shows that compound C1 can inhibit DDR1-mediated lung fibrosis at the cellular level and thus can inhibit fibrotic diseases (such as lung fibrosis).

Compounds C2-C12 can also inhibit the DDR1-mediated lung fibrosis at the cellular level and thus can inhibit fibrotic diseases (such as lung fibrosis).

The preferred embodiments of the present invention have been described in detail above, however, the present invention is not limited to the specific details of the aforementioned embodiments, and various simple modifications can be made to the technical solutions of the present invention within the scope of the technical concept of the present invention and fall within the scope of the present invention.

It is also to be noted that the various specific technical features described in the above detailed description may be combined in any suitable manner without any contradiction. In order to avoid unnecessary repetition, the present invention does not separately describe the various possible combination ways.

In addition, arbitrary combinations of the various embodiments of the present invention are allowed and should be deemed as being disclosed in the present invention as long as they do not violate the idea of the present invention.

1. A compound, wherein the compound is a compound represented by Formula A or a pharmaceutically acceptable salt thereof: 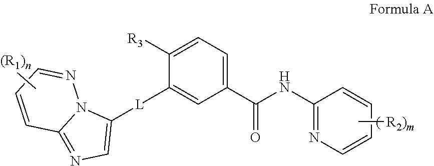
wherein each R₁ is independently alkyl, alkenyl, alkynyl, cycloalkyl, cycloalkenyl, cycloalkynyl, aryl, heterocyclyl or heteroaryl;
n is 1 or 2; m is 0, 1 or 2;
each R₂ is independently alkyl, haloalkyl, alkenyl, alkynyl, cycloalkyl, cycloalkenyl, cycloalkynyl, aryl, heterocyclyl and heteroaryl;
R₃ is methyl, ethyl, n-propyl or isopropyl;
L is ethynylene, phenylene or methylenephenyl.

2. The compound of claim 1, wherein n is 1.

3. The compound of claim 1, wherein R₁ is cycloalkenyl which is cyclopentenyl, methylcyclopentenyl or cyclohexenyl.

4. The compound of claim 3, wherein the cycloalkenyl is cyclohexen-1-yl.

5. The compound of claim 1, wherein m is 0.

6. The compound of claim 5, wherein L is ethynylene.

7. The compound of claim 1, wherein the compound is any one of the compounds shown in the following Formulas C1-C12:

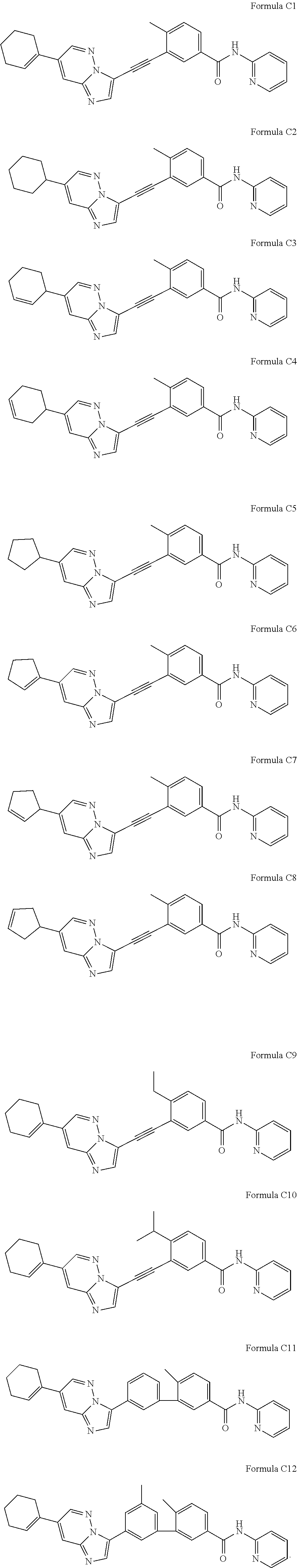

8. A pharmaceutical composition, wherein the pharmaceutical composition comprises a compound of claim 1 and at least one pharmaceutically acceptable excipient.

9. Use of a compound of claim 1 in the manufacture of a medicament for the treatment of a disease, wherein the disease is at least one of inflammation, fibrotic disease and cancer.

10. The use of claim 9, wherein the inflammation is at least one of hepatitis, periostitis and nephritis; the cancer is at least one of head and neck cancer, liver cancer, prostate cancer, lymphoma, leukemia, glioma, lung cancer, brain cancer, and breast cancer; and the fibrotic disease is at least one of liver fibrosis, kidney fibrosis and lung fibrosis.
